SEQUENCE LISTING (55 sequences)

SEQ ID NO: 1 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagaggcctggacaaggccttgagtggatcggagagattgatccttctgatagttatactaactacaatcaaaagttcaagggcaaggccaagttgactgtagacaaatcctccagcacagcctacatggaagtcagcagcctgacatctgaggactctgcggtctattactgtgcaagaggtagcaacccttactactatgctatggactactggggtcaaggaacctcagtcaccgtctcctca

SEQ ID NO: 2 - DNA, 360 bp, synthetic construct
  source 1..360: mol_type = other DNA
caggtgcaactgcagcagcctggggctgaagtggtgaagcctggggcttcagtgagactgtcctgcgagacttctggctacaccttcaccagcaactggatgcactgggtgaagcagaggcctgaccaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 3 - DNA, 360 bp, synthetic construct
  source 1..360: mol_type = other DNA
caggtgcaactgcagcagcctggggctgaagtggtgaagcctggggcttcagtgagactgtcctgcaagacttctggctacaccttcaccagcaactggatgcactgggtgaagcagagacctggacaaggacttgagtggatcggagagattgatccttctgatagttatactaactacaatcaaaagttcaagggcaaggccgaattgactgtagacaaatcctccagcacagcctacatggaagtcagcgacctgacatctgaggactctgcggtctattactgtgcaagaggtagcaacccttactactatgctatggactactggggtcaaggaacctcagtcaccgtctcctca

SEQ ID NO: 4 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgaagcagaggcctggacaaggacttgagtggatcggagagattgatccttctgatagttatactaactacaatcaaaagttcaagggcaaggccaagttgactgtagacaaatcctccagcacagcctacatggaagtcagcgacctgacagctgaggactctgcggtctattactgtgcaagaggtagcaacccttactactatgctatggactactggggtcaaggaacctcagtcaccgtctcctca

SEQ ID NO: 5 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagacgcctggaaaaggacttgagtggatcggagagattgatccttctgatagttatactaactacaatcaaaagttcgatggcaaggccaagttgactgtagacaaatcctccagcacagcctacatggaagtcagcgacctgacagctgaggactctgcgacctattactgtgcaagaggtagcaacccttactactatgctatggactactggggtcaaggaacctcagtcaccgtctcctca

SEQ ID NO: 6 - DNA, 360 bp, synthetic construct
  source 1..360: mol_type = other DNA
caggtgcaactggagcagcctggggctgaagtgaagaagcctggggcttcagtgaaggtgtcctgcaaggcttctggctacaccttcaccagcaactggatgcactgggtgagacaggcacctggaaaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 7 - DNA, 360 bp, synthetic construct
  source 1..360: mol_type = other DNA
caggtgcaactggagcagcctggggctgaagtggtgaagcctggggcttcagtgaaggtgtcctgcaagacttctggctacaccttcaccagcaactggatgcactgggtgaagcagaggcctggacaaggacttgagtggatcggagagattgatccttctgatagttatactaactacaatcaaaagttcgatggcaaggccaagttgactgtagacaaatcctccagcacagcctacatggaagtcagcgacctgacatctgaggactctgcgacctattactgtgcaagaggtagcaacccttactactatgctatggactactggggtcaaggaacctcagtcaccgtctcctca

SEQ ID NO: 8 - DNA, 311 bp, synthetic construct
  source 1..311: mol_type = other DNA
caaattgttctcacccagtctccagcaatcatgtctgcatctccaggggagaaggtcaccatgacctgcagtgccagctcaggtgtcaactacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 9 - DNA, 311 bp, synthetic construct
  source 1..311: mol_type = other DNA
caaattgctctcacccaggagccagcaatcatgtctgcatctccaggggagaaggtcaccatgacctgcagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 10 - DNA, 311 bp, synthetic construct
  source 1..311: mol_type = other DNA
gacattgttctcacccagtctccagcaactttgtctgcatctccaggggagaaggtcaccatgacctgtagtgccagctcaggtgtcaactacatgcactggtatcagcagaagccaggcacctcccccaaaagatggatttatgacacatccaaactggattctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 11 - DNA, 311 bp, synthetic construct
  source 1..311: mol_type = other DNA
caaattgttctcacccagtctccagcaactttgtctgcatctccaggggagaaggctaccatgacctgcagtgccagctcaggtgctaactacatgcactggtaccagcagaagccaggcacctcccccaaaagatggatttatgacacatccaaactggcttctggagtccctgctcgcttcagtggcagtgggtctgggacctcttattctctcacaatcgagagcatggaggctgaagatgctgccacttattactgccatcagcgaggtagttacacgttcggaggggggaccaagctggaaataaa

SEQ ID NO: 12 - DNA, 310 bp, synthetic construct
  source 1..310: mol_type = other DNA
caaattgttctcacccagtctccagcaactttgtctgcatctccaggggagaaggctacatgacctgtagtgccagctcaggtgtcaactacatgcactggtaccagcagaagccaggcacctcccccaaaagatggatttatgacacagacaaaacggcttctggagtccctgctcgcttcagtggcagtgggtctgggacctcttattctctcacaatcagcagcatggaggctgaagatgctgccacttattactgccatcagcgaggtagttacacgttcggaggggggaccaagctggaaataaa

SEQ ID NO: 13 - protein, 120 aa, synthetic construct
QVQLQQPGAEVVKPGASVRLSCKTSGYTFTSNWMHWVKQRPGQGLEWIGEIDPSDSYTNYNQKFKGKAKLTVDKSSSTAYMEVSSLTSEDSAVYYCARGSNPYYYAMDYWGQGTSVTVSS

SEQ ID NO: 14 - protein, 120 aa, synthetic construct
QVQLQQPGAEVVKPGASVRLSCETSGYTFTSNWMHWVKQRPDQGLEWIGEIDPSDSYTNYNQKFKGKAELTVDKSSSTAYMEVSDLTSEDSAVYYCARGSNPYYYAMDYWGQGTSVTVSS

SEQ ID NO: 15 - protein, 120 aa, synthetic construct
QVQLQQPGAEVVKPGASVRLSCKTSGYTFTSNWMHWVKQRPGQGLEWIGEIDPSDSYTNYNQKFKGKAELTVDKSSSTAYMEVSDLTSEDSAVYYCARGSNPYYYAMDYWGQGTSVTVSS

SEQ ID NO: 16 - protein, 120 aa, synthetic construct
QVQLEQPGAEVKKPGASVKVSCKASGYTFTSNWMHWVKQRPGQGLEWIGEIDPSDSYTNYNQKFKGKAKLTVDKSSSTAYMEVSDLTAEDSAVYYCARGSNPYYYAMDYWGQGTSVTVSS

SEQ ID NO: 17 - protein, 120 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 18 - protein, 120 aa, synthetic construct
QVQLEQPGAEVKKPGASVKVSCKASGYTFTSNWMHWVRQAPGKGLEWIGEIDPSDSYTNYNQKFDGKAKLTVDKSSSTAYMEVSDLTSEDSATYYCARGSNPYYYAMDYWGQGTSVTVSS

SEQ ID NO: 19 - protein, 120 aa, synthetic construct
QVQLEQPGAEVVKPGASVKVSCKTSGYTFTSNWMHWVKQRPGQGLEWIGEIDPSDSYTNYNQKFDGKAKLTVDKSSSTAYMEVSDLTSEDSATYYCARGSNPYYYAMDYWGQGTSVTVSS

SEQ ID NO: 20 - protein, 120 aa, synthetic construct
QVQLQQPGAEVKKPGASVRLSCETSGYTFTSNWMHWVKQRPDQGLEWIGEIDPSDSYTNYNQKFDGKAELTVDKSSSTAYMEVSDLTSEDSAVYYCARGSNPYYYAMDYWGQGTSVTVSS

SEQ ID NO: 21 - protein, 120 aa, synthetic construct
QVQLEQPGAEVKKPGASVKVSCKASGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 22 - protein, 29 aa, synthetic construct
QVQLXQPGAEVXKPGASVXXSCXXSGYTF

SEQ ID NO: 23 - protein, 14 aa, synthetic construct
WVXQXPXXGLEWIG

SEQ ID NO: 24 - protein, 38 aa, synthetic construct
YNQKFXGKAXLTVDKSSSTAYMEVSXLTXEDSAXYYCA

SEQ ID NO: 25 - protein, 104 aa, synthetic construct
QIVLTQSPAIMSASPGEKVTMTCSASSGVNYMHWYQQKPGTSPKRWIYDTSKLASGVPARFSGSGSGTSYSLTISSMEAEDAATYYCHQRGSYTFGGGTKLEIK

SEQ ID NO: 26 - protein, 104 aa, synthetic construct
QIALTQEPAIMSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 27 - protein, 104 aa, synthetic construct
DIVLTQSPATLSASPGEKVTMTCSASSGVNYMHWYQQKPGTSPKRWIYDTSKLDSGVPARFSGSGSGTSYSLTISSMEAEDAATYYCHQRGSYTFGGGTKLEIK

SEQ ID NO: 28 - protein, 104 aa, synthetic construct
QIVLTQSPATLSASPGEKATMTCSASSGANYMHWYQQKPGTSPKRWIYDTSKLASGVPARFSGSGSGTSYSLTIESMEAEDAATYYCHQRGSYTFGGGTKLEIK

SEQ ID NO: 29 - protein, 104 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 30 - protein, 104 aa, synthetic construct
QIALTQEPAIMSASPGEKATMTCSASSGVNYMHWYQQKPGTSPKRWIYDTSKLDSGVPARFSGSGSGTSYSLTIESMEAEDAATYYCHQRGSYTFGGGTKLEIK

SEQ ID NO: 31 - protein, 104 aa, synthetic construct
QIVLTQSPATLSASPGEKATMTCSASSGANYMHWYQQKPGTSPKRWIYDTDKTASGVPARFSGSGSGTSYSLTIESMEAEDAATYYCHQRGSYTFGGGTKLEIK

SEQ ID NO: 32 - protein, 23 aa, synthetic construct
XIXLTQXPAXXSASPGEKXTMTC

SEQ ID NO: 33 - skipped

SEQ ID NO: 34 - protein, 120 aa, synthetic construct; identical to SEQ ID NO: 13

SEQ ID NO: 35 - protein, 104 aa, synthetic construct; identical to SEQ ID NO: 25

SEQ ID NO: 36 - protein, 9 aa, synthetic construct
VRLSCKTSG

SEQ ID NO: 37 - protein, 9 aa, synthetic construct
WVKQRPGQG

SEQ ID NO: 38 - protein, 9 aa, synthetic construct
YNQKFKGKA

SEQ ID NO: 39 - protein, 9 aa, synthetic construct
FKGKAKLTV

SEQ ID NO: 40 - protein, 9 aa, synthetic construct
YMEVSSLTS

SEQ ID NO: 41 - protein, 9 aa, synthetic construct
VYYCARGSN

SEQ ID NO: 42 - protein, 9 aa, synthetic construct
IVLTQSPAI

SEQ ID NO: 43 - protein, 9 aa, synthetic construct
VLTQSPAIM

SEQ ID NO: 44 - protein, 9 aa, synthetic construct
VTMTCSASS

SEQ ID NO: 45 - protein, 9 aa, synthetic construct
VNYMHWYQQ

SEQ ID NO: 46 - protein, 9 aa, synthetic construct
WIYDTSKLA

SEQ ID NO: 47 - protein, 9 aa, synthetic construct
IYDTSKLAS

SEQ ID NO: 48 - protein, 13 aa, synthetic construct
CGTAAGTGGATCC

SEQ ID NO: 49 - protein, 12 aa, synthetic construct
GCTAGCTCCAGC

SEQ ID NO: 50 - protein, 450 aa, synthetic construct
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 450 aa, synthetic construct; identical to SEQ ID NO: 50

SEQ ID NO: 52 - protein, 211 aa, synthetic construct
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 211 aa, synthetic construct; identical to SEQ ID NO: 52

SEQ ID NO: 54 - protein, 450 aa, synthetic construct; identical to SEQ ID NO: 50

SEQ ID NO: 55 - protein, 211 aa, synthetic construct; identical to SEQ ID NO: 52

(19) United States
(12) Patent Application Publication
(10) Pub. No.: US 2026/0234249 A1
(43) Pub. Date: Aug. 13, 2026

(54) METHODS FOR PREPARATION OF ANTI-CD19 ANTIBODIES

(71) Applicant: Climb Bio Operating Inc., Wellesley, MA (US)

(72) Inventors: Tatyana Touzova, Scotch Plains, NJ (US); Stephen Thomas, Wellesley, MA (US); Nathalie Rigal, Berlin (DE); Sascha Ramm, Berlin (DE); Susanne Seitz, Berlin (DE)

(21) Appl. No.: 19/355,258

(22) Filed: Oct. 10, 2025

Related U.S. Application Data
(60) Provisional application No. 63/706,967, filed on Oct. 14, 2024.

(51) Int. Cl. C07K 16/28

(52) U.S. Cl. CPC C07K 16/2803; C07K 2317/51; C07K 2317/515

(57) ABSTRACT

The present invention is directed to anti-CD19 antibodies and methods of preparing the anti-CD19 antibodies for treatment of diseases.

### I. FIELD OF THE INVENTION

The invention relates to the methods for preparation of preferred anti-CD19 antibodies in Chinese Hamster Ovary (CHO) cells.

### II. SEQUENCE LISTING

The present application is being filed along with a Sequence Listing XML in electronic format. The Sequence Listing XML is provided as a file named CLIMB-009-01US-Seq-Listing.xml, created on Oct. 9, 2025, which is 55,938 bytes in size. The Sequence Listing XML file and the information in the electronic format of the Sequence Listing XML file is incorporated herein by reference in its entirety.

### III. BACKGROUND

CD19 is a surface protein found on B cells and on certain cancerous cells derived from B cells, such as many B cell lymphomas. Anti-CD19 monoclonal antibodies have been generated in mice, and show some promise in pre-clinical animal models of B cell-derived cancers. However, mouse-derived antibodies are generally immunogenic in humans. A number of strategies have been developed to alter mouse-derived antibodies to minimize their immunogenicity in humans. One such strategy, chimerization, involves the fusion of mouse variable regions to human constant regions. However, the mouse-derived variable region sequences remaining following chimerization will often be immunogenic. Another such strategy, humanization, involves the replacement of mouse-derived framework regions (FRs) within the variable regions with the most closely related human-derived sequences, with the optional reversion of certain amino acids back to the corresponding mouse amino acid in order to maintain binding activity. However, even humanized antibodies may be immunogenic, since the antibody complementarity determining regions (CDRs) generally contain B cell epitopes and T cell epitopes that are non-self. Indeed, even fully human antibodies are immunogenic; this is the basis for the formation of anti-idiotype antibodies during the course of an immune response. All of these problems may apply to mouse-derived anti-CD19 antibodies as they would to any other type of antibody. Therefore, there is a need for anti-CD19 antibodies with reduced immunogenicity.

### IV. SUMMARY OF THE INVENTION

Although the anti-CD19 antibodies with reduced immunogenicity are required for the treatment of patients with those associated conditions, the conventional methods for production of antibodies have some significant shortcomings, including the scalability of such processes.

The invention provides methods of producing anti-CD19 antibodies. The invention recognizes that the anti-CD19 antibodies will have to be produced at commercially viable scale, especially if an anti-CD19 antibody is of therapeutic interest for the patients. Accordingly, the present invention is directed to a method or producing an anti-CD19 antibody. In certain aspects, the present invention provides methods and processes for manufacturing the anti-CD19 antibodies in commercially viable scales.

In certain aspects, the invention provides a process for preparation of an anti-CD19 antibody, wherein: the anti-CD19 antibody comprises variable domain comprising a heavy chain variable region that is at least 90% identical to SEQ ID NO: 13 and has an amino acid substitution at one or more residues corresponding to Gln5, Arg19, Leu20, Arg40, Gln43, Lys65, Ser85, Ser88, and Val93; and the process comprises production of the anti-CD19 antibody in Chinese Hamster Ovary (CHO) cells.

In certain aspects, the invention provides a process for preparation of anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the process comprises production of the anti-CD19 antibody in Chinese Hamster Ovary (CHO) cells.

In certain aspects, the invention provides a process for preparation of an anti-CD19 antibody, wherein: the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and the process comprises production of the anti-CD19 antibody in Chinese Hamster Ovary (CHO) cells.

In certain embodiments, the anti-CD19 antibody variable domain comprises a heavy chain variable region of SEQ ID NO: 17 and a light chain variable region of SEQ ID NO: 29. In certain embodiments, the anti-CD19 antibody is provided in U.S. Pat. No. 8,691,952, which is incorporated by reference in its entirety. In certain embodiments, the anti-CD19 antibody variable domain comprises a heavy chain variable region of SEQ ID NO: 17 and a light chain variable region of SEQ ID NO: 29 are provided in U.S. Pat. No. 8,691,952.

In certain embodiments, the heavy chain variable region of the anti-CD19 antibody has, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, the heavy chain variable region of the anti-CD19 antibody is SEQ ID NO: 13 comprising an amino acid substitution at one or more residues corresponding to Gln5, Arg19, Leu20, Arg40, Gln43, Lys65, Ser85, Ser88, and Val93.

In certain embodiments, the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody is the amino acid sequence of SEQ ID NO: 17.

In certain embodiments, the anti-CD19 antibody variable domain of the anti-CD19 antibody further comprises a light chain variable region that is at least 90% identical to SEQ ID NO: 25 and has an amino acid substitution at one or more residues corresponding to Ile10, Met11, Val19, Ser51, and Leu53.

In certain embodiments, wherein the light chain variable region the anti-CD19 antibody has one or more amino acid substitutions selected from the group consisting of Ile10Thr, Met11Leu, Val19Ala, Ser51Asp, and Leu53Thr.

In certain embodiments, the light chain variable region is at least 95% identical to SEQ ID NO: 25.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is SEQ ID NO: 25 comprising an amino acid substitution at one or more residues corresponding to Ile10, Met11, Val19, Ser51, and Leu53.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is the amino acid sequence of SEQ ID NO: 29.

In certain embodiments, the heavy chain variable region comprises one or more of substitutions Gln5Glu, Arg19Lys, Leu20Val, Arg40Thr, Gln43Lys, Lys65Asp, Ser85Asp, Ser88Ala, and Val93Thr.

In certain embodiments, wherein the light chain variable region comprises one or more of substitutions Ile10Thr, Met11Leu, Val19Ala, Ser51Asp, and Leu53Thr.

In certain embodiments, the anti-CD19 antibody is VB119. In certain embodiments, VB119 is an anti-CD19, IgG1 monoclonal antibody. VB119 demonstrates antibody-dependent cell mediated toxicity (ADCC) with minimal to no complement dependent cytotoxicity (CDC).

In certain embodiments, the anti-CD19 antibody comprises the below listed subunits in the anti-CD19 antibody:

Subunit 1 (SEQ ID NO: 50):
QVQLEQPGAEVVKPGASVKVSCKTSGYTFTSNWMHWVKQTPGKGLEWIG
EIDPSDSYTNYNQKFDGKAKLTVDKSSSTAYMEVSDLTAEDSATYYCAR
GSNPYYYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Subunit 2 (SEQ ID NO: 51):
QVQLEQPGAEVVKPGASVKVSCKTSGYTFTSNWMHWVKQTPGKGLEWIG
EIDPSDSYTNYNQKFDGKAKLTVDKSSSTAYMEVSDLTAEDSATYYCAR
GSNPYYYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Subunit 3 (SEQ ID NO: 52):
QIVLTQSPATLSASPGEKATMTCSASSGVNYMHWYQQKPGTSPKRWIYD
TDKTASGVPARFSGSGSGTSYSLTISSMEAEDAATYYCHQRGSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC
Subunit 4 (SEQ ID NO: 53):
QIVLTQSPATLSASPGEKATMTCSASSGVNYMHWYQQKPGTSPKRWIYD
TDKTASGVPARFSGSGSGTSYSLTISSMEAEDAATYYCHQRGSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC
In certain embodiments, the heavy chain sequence
of the anti-CD19 antibody is provided
below (SEQ ID NO: 54):
QVQLEQPGAEVVKPGASVKVSCKTSGYTFTSNWMHWVKQTPGKGLEWIG
EIDPSDSYTNYNQKFDGKAKLTVDKSSSTAYMEVSDLTAEDSATYYCAR
GSNPYYYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
In certain embodiments, the light chain sequence
of the anti-CD19 antibody is provided
below (SEQ ID NO: 55):
QIVLTQSPATLSASPGEKATMTCSASSGVNYMHWYQQKPGTSPKRWIYD
TDKTASGVPARFSGSGSGTSYSLTISSMEAEDAATYYCHQRGSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC

In certain embodiments, the anti-CD19 antibody variable domain comprises a heavy chain variable region of SEQ ID NO: 17 and a light chain variable region of SEQ ID NO: 29. In certain instances, the anti-CD19 antibody is budoprutug. In certain embodiments, budoprutug is an anti-CD19 antibody variable domain comprises a heavy chain variable region of SEQ ID NO: 17 and a light chain variable region of SEQ ID NO: 29.

In certain embodiments, the CHO cells in methods of the invention are CHO DG44 cells. In certain embodiments, the CHO DG44 cells advantageously provide optimal production of the anti-CD19 antibodies. In certain beneficial aspects, the methods of the invention do not involve the use of live viruses and/or infectious retroviral particles.

In certain embodiments, the methods of the invention comprise one or more of the below steps:
thawing the CHO cells;
culturing the thawed CHO cells;
harvest the cultured CHO cells;
isolate the anti-CD19 antibody; and
purify the anti-CD19 antibody.

Advantageously, the processes of the invention, wherein the anti-CD19 antibody is produced by CHO cells is advantageous as compared to the processes wherein said antibody is produced by YB2/0 cells. In particular, the invention recognized that the processes involving the production of anti-CD19 antibodies in YB2/0 cells, would have been unable to prepare the quantities in commercially viable scales.

In contrast, advantageously, the methods of the invention allow for generation of higher protein expression per cell of anti-CD19 antibodies. In certain preferred embodiments, the methods of the invention lead to production of greater than 3.0 g/L titer of anti-CD19 antibodies. In certain preferred embodiments, the invention beneficially allows for the production of 250 L batches of anti-CD19 antibodies. In certain embodiments, the methods of the invention allows for the production of batches of 250 L or greater with a titer of 3.0 g/L or higher titer of anti-CD19 antibodies.

In another beneficial aspect of the invention, the invention provides that even though the anti-CD19 antibody is produced using a new cost efficient and scalable for commercial production, the anti-CD19 antibodies has similar biological activity as compared to the anti-CD19 antibodies produced by other methods. In certain preferred aspects, the biological activity of the anti-CD19 antibodies produced by methods of the invention has biological activity of at least ~75% as compared to the anti-CD19 antibodies produced by other methods.

Importantly, in certain embodiments, the invention beneficially allows for the control of Fc-fucosylation levels of the antibody. Modulating the levels of the fucosylation of the antibody has a significant impact on the biological activity of the said antibody.

In the processes involving the production of anti-CD19 antibodies in YB2/0 cells, the modulation of fucosylation of the produced antibody was challenging. In particular, the YB2/0 cells have reduced fucosylation in the Fc region because the reduced expression of fructosyltransferase enzyme (FUT8). The level of fucosylation is an important parameter for the anti-CD19 antibodies and it impacts the biological activity of the anti-CD19 antibodies.

In comparison, the methods of the invention advantageously provide an optimal fucosylation pattern of the product. In certain beneficial aspects, the optimal fucosylation product of anti-CD19 antibodies, including VB119 have biological activity.

In certain embodiments, the methods of the invention rely on production of anti-CD19 antibodies using a technology for optimal production and fucosylation of antibodies. In certain embodiments, the technology provides heterologous, stable, cytosolic expression of the bacterial enzyme GDP-4keto-6-deoxy-D-mannose reductase (RMD) in a cell that is intended to be used for production of a glycoprotein or an antibody by subsequent (or simultaneous) transduction of respective genes of the glycoprotein/antibody.

Beneficially, RMD redirects the de-novo fucose synthesis pathway towards a sugar-nucleotide (GDP-Rhamnose) that cannot be metabolized by the cell and moreover acts as an inhibitor of the pathway. Even lowest levels expression of this enzyme is sufficient to block the de novo fucose synthetic pathway almost completely. In certain embodiments, the technology used by methods of the invention is provided included in U.S. Pat. Nos. 7,579,170, 7,931,895, 7,541,029, 8,124,078, 8,153,124, 8,178,093, 8,409,572, 8,642,292, and 8,357,370 which are incorporated by reference in its entirety.

### V. BRIEF DESCRIPTION OF THE DRAWINGS

FIG. 1 provides ADCC data comparison for budoprutug and low-fucose budoprutug as compared to wt-IgG.

### VI. DETAILED DESCRIPTION

The present invention is directed to processes for preparation of an anti-CD19 murine monoclonal B4 antibody which has been modified to reduce its immunogenicity in comparison to wild-type B4 antibody. More specifically, the variable region of the B4 antibody of the invention is modified to remove potential T-cell epitopes. As a result, B4 antibodies produced by the methods of the invention have improved biological properties compared to wild-type B4 antibodies. More specifically, the mutations within a B4 antibody that have the effect of reducing the immunogenicity of a B4 antibody itself, primarily by removing T-cell epitopes within B4 that may stimulate to an immune response.

### Anti-CD19 Antibodies of the Invention:

In certain aspects, the invention provides methods of preparing anti-CD19 antibodies which feature an amino acid sequence defining a modified immunoglobulin heavy chain framework region comprising amino acid residues 1-30 of SEQ ID NO:22, wherein one or more of the amino acid residues at positions X5, X12, X19, X20, X23, and X24 are as follows: X5 is Q or E, X12 is V or K, X19 is R or K, X20 is L or V, X23 is K, E or D, or X24 is T or A. In certain embodiments, at least one of the amino acid residues at positions X5, X12, X19, X20, X23, or X24 is not the same amino acid residue as the amino acid at the corresponding position in the unmodified immunoglobulin heavy chain framework region as set forth in amino acid residues 1-30 of SEQ ID NO:13. In one embodiment, X23 is E or D.

In certain aspects, the invention provides methods of preparing anti-CD19 antibodies which feature an amino acid sequence defining a modified immunoglobulin heavy chain framework region comprising amino acid residues 1-14 of SEQ ID NO:23, wherein one or more of the amino acid residues at positions X3, X5, X7, and X8, are as follows: X3 is K or R, X5 is R, T, or A, X7 is G, D, or E, or X8 is Q or K. According to this aspect of the invention, at least one of the amino acid residues at positions X3, X5, X7, or X8 is not the same as the amino acid at the corresponding position in the unmodified immunoglobulin heavy chain framework region as set forth in amino acid residues 36-49 of SEQ ID NO:13. In one embodiment, X7 is E or D.

In certain aspects, the invention provides methods of preparing anti-CD19 antibodies which feature an amino acid sequence defining a modified immunoglobulin heavy chain framework region comprising amino acid residues 1-39 of SEQ ID NO:24, wherein one or more of the amino acid residues at positions X6, X10, X26, X29, and X34 are as follows: X6 is K, D, or E, X10 is K, E, or D, X26 is S, D, or E, X29 is S or A, or X34 is V or T. According to this aspect of the invention, at least one of the amino acid residues at positions X6, X10, X26, X29, or X34 is not the same as the amino acid at the corresponding position in the unmodified immunoglobulin heavy chain framework region as set forth in amino acid residues 60-98 of SEQ ID NO:13. In one embodiment, X10 is E or D.

In certain aspects, the invention provides methods of preparing anti-CD19 antibodies which feature an amino acid sequence defining a modified immunoglobulin light chain framework region comprising amino acid residues 1-23 of SEQ ID NO:32, wherein one or more of the amino acid residues at positions X1, X3, X7, X10, X11, and X19 are as follows: X1 is Q or D, X3 is V or A, X7 is S or E, X10 is I or T, X11 is M or L, or X19 is V or A. According to this aspect of the invention, at least one of the amino acid residues at positions X1, X3, X7, X10, X11, or X19 is not the same as the amino acid at the corresponding position in the unmodified immunoglobulin light chain framework region as set forth in amino acid residues 1-23 of SEQ ID NO:25. In one embodiment, X3 is A and X7 is E. In another embodiment, X1 is D, X10 is I, and X11 is L.

In another aspect, the invention features an amino acid sequence defining a modified immunoglobulin light chain complementarity determining region comprising amino acid residues 24-33 of SEQ ID NO:28.

In another aspect, the invention features an amino acid sequence defining a modified immunoglobulin light chain framework region comprising amino acid residues 56-87 of SEQ ID NO: 28.

According to another aspect, the invention features an antibody variable region comprising an amino acid sequence selected from the group consisting of SEQ ID NO: 14, SEQ ID NO: 15, SEQ ID NO: 16, SEQ ID NO: 17, SEQ ID NO: 20, SEQ ID NO: 21, SEQ ID NO: 26, SEQ ID NO: 27, SEQ ID NO: 28, SEQ ID NO: 29, SEQ ID NO: 30, or SEQ ID NO: 31, wherein the antibody variable region specifically binds to CD19.

According to another aspect, the invention features a polypeptide at least 90% or at least 95% identical to a B4 antibody heavy chain variable region, the polypeptide comprising an amino acid substitution at one or more residues corresponding to Val12, Leu20, Lys23, Thr24, Lys38, Gly42, Gln43, Lys65, Lys69, Ser85, Ser88, or Val93. In one embodiment, the polypeptide comprises one or more of substitutions Gln5Glu, Val12Lys, Arg19Lys, Leu20Val, Lys23Glu, Lys23Asp, Thr24Ala, Lys38Arg, Arg40Thr, Gly42Asp, Gly42Glu, Gln43Lys, Lys65Asp, Lys65Glu, Lys69Glu, Lys69Asp, Ser85Asp, Ser85Glu, Ser88Ala, or Val93Thr.

According to another aspect, the invention features a polypeptide at least 90% or at least 95% identical to a B4 antibody light chain variable region, the polypeptide comprising an amino acid substitution at one or more residues corresponding to Val3, Ser7, Ile10, Met11, Val19, Val29, Ser51, Leu53, Ala54, or Ser75. In one embodiment, the polypeptide comprises one or more of substitutions Gln1Asp, Val3Ala, Ser7Glu, Ile10Thr, Met11Leu, Val19Ala, Val29Ala, Ser51Asp, Leu53Thr, Ala54Asp, or Ser75Glu.

In another aspect, the invention features a method of treating a patient, the method comprising the step of administering a therapeutically effective amount of a anti-CD19 prepared by methods provided in the invention.

In another aspect, the invention features a method for targeting a cell with CD19 on its surface, the method comprising the step of administering an antibody variable region according to any one of the embodiments of the invention. In one embodiment of the method the cell is a tumor cell.

In certain aspects, the invention provides a process for preparation of anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the process comprises production of the anti-CD19 antibody in Chinese Hamster Ovary (CHO) cells.

In certain aspects, the invention provides a process for preparation of an anti-CD19 antibody, wherein: the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and the process comprises production of the anti-CD19 antibody in Chinese Hamster Ovary (CHO) cells.

In certain embodiments, the anti-CD19 antibody variable domain comprises a heavy chain variable region of SEQ ID NO: 17 and a light chain variable region of SEQ ID NO: 29. In certain embodiments, the anti-CD19 antibody is provided in U.S. Pat. No. 8,691,952, which is incorporated by reference in its entirety. In certain embodiments, the anti-CD19 antibody variable domain comprises a heavy chain variable region of SEQ ID NO: 17 and a light chain variable region of SEQ ID NO: 29 are provided in U.S. Pat. No. 8,691,952.

In certain embodiments, the heavy chain variable region of the anti-CD19 antibody has, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, the heavy chain variable region of the anti-CD19 antibody is SEQ ID NO: 13 comprising an amino acid substitution at one or more residues corresponding to Gln5, Arg19, Leu20, Arg40, Gln43, Lys65, Ser85, Ser88, and Val93.

In certain embodiments, the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

In certain embodiments, wherein the heavy chain variable region of the anti-CD19 antibody is the amino acid sequence of SEQ ID NO: 17.

In certain embodiments, the anti-CD19 antibody variable domain of the anti-CD19 antibody further comprises a light chain variable region that is at least 90% identical to SEQ ID NO: 25 and has an amino acid substitution at one or more residues corresponding to Ile10, Met11, Val19, Ser51, and Leu53.

In certain embodiments, wherein the light chain variable region the anti-CD19 antibody has one or more amino acid substitutions selected from the group consisting of Ile10Thr, Met11Leu, Val19Ala, Ser51Asp, and Leu53Thr.

In certain embodiments, the light chain variable region is at least 95% identical to SEQ ID NO: 25.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is SEQ ID NO: 25 comprising an amino acid substitution at one or more residues corresponding to Ile10, Met11, Val19, Ser51, and Leu53.

In certain embodiments, the light chain variable region has one or more amino acid substitutions selected from the group consisting of Ile10Thr, Met11Leu, Val19Ala, Ser51Asp, and Leu53Thr.

In certain embodiments, the light chain variable region is the amino acid sequence of SEQ ID NO: 29.

In certain embodiments, the heavy chain variable region comprises one or more of substitutions Gln5Glu, Arg19Lys, Leu20Val, Arg40Thr, Gln43Lys, Lys65Asp, Ser85Asp, Ser88Ala, and Val93Thr.

In certain embodiments, wherein the light chain variable region comprises one or more of substitutions Ile10Thr, Met11Leu, Val19Ala, Ser51Asp, and Leu53Thr.

In certain embodiments, the anti-CD19 antibody is VB119. In certain embodiments, VB119 is an anti-CD19, IgG1 monoclonal antibody. VB119 demonstrates antibody-dependent cell mediated toxicity (ADCC) with minimal to no complement dependent cytotoxicity (CDC).

In certain embodiments, the anti-CD19 antibody comprises one or more of the below listed subunits in the anti-CD19 antibody:

Subunit 1 (SEQ ID NO: 50):
QVQLEQPGAEVVKPGASVKVSCKTSGYTFTSNWMHWVKQTPGKGLEWIG
EIDPSDSYTNYNQKFDGKAKLTVDKSSSTAYMEVSDLTAEDSATYYCAR
GSNPYYYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Subunit 2 (SEQ ID NO: 51):
QVQLEQPGAEVVKPGASVKVSCKTSGYTFTSNWMHWVKQTPGKGLEWIG
EIDPSDSYTNYNQKFDGKAKLTVDKSSSTAYMEVSDLTAEDSATYYCAR
GSNPYYYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Subunit 3 (SEQ ID NO: 52):
QIVLTQSPATLSASPGEKATMTCSASSGVNYMHWYQQKPGTSPKRWIYD
TDKTASGVPARFSGSGSGTSYSLTISSMEAEDAATYYCHQRGSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC
Subunit 4 (SEQ ID NO: 53):
QIVLTQSPATLSASPGEKATMTCSASSGVNYMHWYQQKPGTSPKRWIYD
TDKTASGVPARFSGSGSGTSYSLTISSMEAEDAATYYCHQRGSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC
In certain embodiments, the heavy chain sequence
of the anti-CD19 antibody is provided
below (SEQ ID NO: 54):
QVQLEQPGAEVVKPGASVKVSCKTSGYTFTSNWMHWVKQTPGKGLEWIG
EIDPSDSYTNYNQKFDGKAKLTVDKSSSTAYMEVSDLTAEDSATYYCAR
GSNPYYYAMDYWGQGTSVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
In certain embodiments, the light chain sequence
of the anti-CD19 antibody is provided
below (SEQ ID NO: 55):
QIVLTQSPATLSASPGEKATMTCSASSGVNYMHWYQQKPGTSPKRWIYD
TDKTASGVPARFSGSGSGTSYSLTISSMEAEDAATYYCHQRGSYTFGGG
TKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC

In certain embodiments, the anti-CD19 antibody is budoprutug. In certain embodiments, budoprutug comprises a heavy chain and a light chain. In certain embodiments, budoprutug comprises heavy chain and light chain provided in SEQ ID NO: 54 and SEQ ID NO: 55 respectively.

In certain embodiments, the heavy chain of anti-CD19 antibody is at least 90% identical to SEQ ID NO: 54. In certain embodiments, the heavy chain of anti-CD19 antibody is at least 95% identical to SEQ ID NO: 54. In certain embodiments, the heavy chain of anti-CD19 antibody is at least 98% identical to SEQ ID NO: 54. In certain embodiments, the heavy chain of anti-CD19 antibody is identical to SEQ ID NO: 54.

In certain embodiments, the light chain of anti-CD19 antibody is at least 90% identical to SEQ ID NO: 55. In certain embodiments, the light chain of anti-CD19 antibody is at least 95% identical to SEQ ID NO: 55. In certain embodiments, the light chain of anti-CD19 antibody is at least 98% identical to SEQ ID NO: 55. In certain embodiments, the light chain of anti-CD19 antibody is identical to SEQ ID NO: 55.

### Fc Portion

In certain embodiments, the antibody variable domains of the antibodies prepared using the methods of the invention are optionally fused to an Fc portion. As used herein, the Fc portion encompasses domains derived from the heavy chain constant region of an immunoglobulin, preferably a human immunoglobulin, including a fragment, analog, variant, mutant or derivative of the constant region. The constant region of an immunoglobulin heavy chain is defined as a naturally-occurring or synthetically produced polypeptide homologous to at least a portion of the C-terminal region of the heavy chain, including the CH1, hinge, CH2, CH3, and, for some heavy chain classes, CH4 domains. The “hinge” region joins the CH1 domain to the CH2-CH3 region of an Fc portion. The constant region of the heavy chains of all mammalian immunoglobulins exhibit extensive amino acid sequence similarity.

In the present invention, the Fc portion typically includes at least a CH2 domain. For example, the Fc portion can include the entire immunoglobulin heavy chain constant region (CH1-hinge-CH2-CH3). Alternatively, the Fc portion can include all or a portion of the hinge region, the CH2 domain and the CH3 domain.

The constant region of an immunoglobulin is responsible for many important antibody effector functions, including those mediated by Fc receptor (FcR) binding and by complement binding. There are five major classes of the heavy chain constant region, classified as IgA, IgG, IgD, IgE, and IgM, each with characteristic effector functions designated by isotype.

IgG, for example, is separated into four γ isotypes: γ₁, γ₂, γ₃, and γ₄, also known as IgG1, IgG2, IgG3, and IgG4, respectively. IgG molecules can interact with multiple classes of cellular receptors including three classes of Fcγ receptors (FcγR) specific for the IgG class of antibody, namely FcγRI, FcγRII, and FcγRIII. The sequences important for the binding of IgG to the FcγR receptors have been reported to be in the CH2 and CH3 domains.

It is also often useful to alter the serum half-life of the antibody. The serum half-life of an antibody, as of an immunoglobulin fusion protein, is influenced by the ability of that antibody to bind to an Fc receptor (FcR) (Gillies et al., Cancer Research (1999) 59:2159-66). The CH2 and CH3 domains of IgG2 and IgG4 have undetectable or reduced binding affinity to Fc receptors compared to those of IgG1. Accordingly, the serum half-life of the featured antibody can be increased by using the CH2 and/or CH3 domain from IgG2 or IgG4 isotypes. Alternatively, the antibody can include a CH2 and/or CH3 domain from IgG1 or IgG3 with modification in one or more amino acids in these domains to reduce the binding affinity for Fc receptors (see, e.g., U.S. patent application Ser. No. 09/256,156, published as U.S. patent application publication 2003-0105294).

In certain embodiments, an Fc portion fused to an antibody variable region of the invention can contain CH2 and/or CH3 domains and a hinge region that are derived from different antibody isotypes. For example, the Fc portion can contain CH2 and/or CH3 domains of IgG2 or IgG4 and a hinge region of IgG1. Assembly of such hybrid Fc portions has been described in U.S. patent application publication 2003-0044423.

When fused to an antibody variable region of the invention, the Fc portion may contain one or more amino acid modifications that generally extend the serum half-life of an Fc fusion protein. Such amino acid modifications include mutations substantially decreasing or eliminating Fc receptor binding or complement fixing activity. For example, one type of such mutation removes the glycosylation site of the Fc portion of an immunoglobulin heavy chain. In IgG1, the glycosylation site is Asn297 (see, for example, U.S. patent application Ser. No. 10/310,719, published as U.S. patent application publication 2003-0166163).

### Methods of Production of the Anti-CD19 Antibodies:

The contemporary methods of production of antibodies leads to a limited titer of antibodies and a low quantity of the antibody being produced. In certain aspects, the present invention provides methods for preparation of anti-CD19 antibodies. In certain embodiments, the methods of preparation of anti-CD19 antibodies result in production of anti-CD19 antibodies that could be manufactured efficiently and maintain the pertinent ADCC profile responsible for the potency of anti-CD19 antibodies.

The invention recognizes that the anti-CD19 antibodies will have to be produced at commercially viable scale, especially if an anti-CD19 antibody is of therapeutic interest for the patients. Accordingly, the present invention is directed to a method or producing an anti-CD19 antibody monoclonal antibody. In certain aspects, the present invention provides methods and processes for manufacturing the anti-CD19 antibodies in commercially viable scales.

The invention provides that the selection of cell lines for biopharmaceutical production is a pivotal decision in the development process, as it profoundly impacts product quality, efficiency, and safety of the anti-CD19 antibody. The invention beneficially provides that production of the YB2/0 cell line has limitations for producing the anti-CD19 antibodies of the invention. In contrast, production of anti-CD19 antibodies enhances the quality and quantity of anti-CD19 antibodies for the patients.

In certain aspects, the invention provides a process for preparation of an anti-CD19 antibody, wherein: the anti-CD19 antibody comprises variable domain comprising a heavy chain variable region that is at least 90% identical to SEQ ID NO: 13 and has an amino acid substitution at one or more residues corresponding to Gln5, Arg19, Leu20, Arg40, Gln43, Lys65, Ser85, Ser88, and Val93; and the process comprises production of the anti-CD19 antibody in Chinese Hamster Ovary (CHO) cells.

Advantageously, the processes of the invention, wherein the anti-CD19 antibody is produced by CHO cells is advantageous over processes of the invention wherein the antibody is produced by YB2/0 cells. In particular, the invention recognized that the processes involving the production of anti-CD19 antibodies in YB2/0 cells, would have been unable to prepare the quantities in commercially viable scales.

In contrast, advantageously, the methods of the invention allow for generation of higher protein expression of anti-CD19 antibodies. In certain preferred embodiments, the methods of the invention lead to production of greater than 3.0 g/L titer of anti-CD19 antibodies. In certain preferred embodiments, the invention beneficially allows for the production of 250 L batches of anti-CD19 antibodies. In certain embodiments, the methods of the invention allow for the production of batches of 250 L or greater with a titer of 3.0 g/L or higher titer of anti-CD19 antibodies. In other beneficial aspects of the invention, the invention provides that even though the anti-CD19 antibody is produced using a new cost efficient and scalable for commercial production, the anti-CD19 antibodies has similar biological activity as compared to the anti-CD19 antibodies produced by other methods. In certain preferred aspects, the biological activity of the anti-CD19 antibodies produced by methods of the invention has biological activity of at least ~95% as compared to the anti-CD19 antibodies produced by other methods.

In certain embodiments, the CHO cells in methods of the invention are CHO DG44 cells. In certain embodiments, the CHO DG44 cells advantageously provide optimal production of the anti-CD19 antibodies. In certain beneficial aspects, the methods of the invention do not involve the use of live viruses and/or infectious retroviral particles.

In certain embodiments, the methods of the invention comprise one or more of the below steps:
thawing the CHO cells; culturing the thawed CHO cells; harvest the cultured CHO cells; isolate the anti-CD19 antibody; and purify the anti-CD19 antibody.

The table below provides a side-by-side comparison of the process of preparation of anti-CD19 antibodies in YB2/0 and CHO cells are provided below.

| YB2/0 Cell Line Process (Process 1) | CHO DG44 Cell Line Process (Process 2) |
|---|---|
| Upstream | Upstream |
| Initial MCB Thaw | Initial MCB Thaw |
| ↓ | ↓ |
| Inoculum Expansion | Inoculum Expansion |
| ↓ | ↓ |
| Seed Culture | Seed Culture |
| ↓ | ↓ |
| 500 L Production Culture | 250 L Production Culture |
| ↓ | ↓ |
| Harvest and Cell Separation by Depth Filtration | Harvest and Cell Separation by Depth Filtration |
| ↓ | ↓ |
| Downstream | Downstream |
| Protein A Capture/Purification | Protein A Capture/Purification |
| ↓ | ↓ |
| Viral Inactivation (low pH hold) | Viral Inactivation (low pH hold) |
| ↓ | ↓ |
| 0.2 μm Filtration | Depth Filtration (0.1-085 μm) NFF |
| ↓ | 0.2 μm Filtration |
| | ↓ |
| Purification by Anion Exchange | Purification by Cation Exchange |
| Chromatography | Chromatography |
| ↓ | ↓ |
| 0.2 μm Filtration | |
| ↓ | |
| Purification by Mix-Mode (Ceramic | Purification by Multi-Modal Anion Exchange |
| Hydroxyapatite) Chromatography | (MMA) |
| ↓ | ↓ |
| 0.2 μm Filtration | |
| ↓ | |
| Concentration and Diafiltration into final | Viral Filtration |
| Formulation Buffer UF/DF | |
| ↓ | ↓ |
| Viral Filtration | Concentration and Diafiltration into final |
| | Formulation Buffer TFF |
| ↓ | ↓ |
| 0.2 μm Filtration | 0.2 μm Filtration |
| ↓ | ↓ |
| DS Packaging and Storage at 2-8 C. | DS Packaging and Storage at ≤−60° C. |

As highlighted in the above table, the CHO DG44 cells are free of adventitious agents as compared to the process of the YB2/0 cells. In certain embodiments, according to the methods of the invention, the medium to culture CHO DG44 cells comprises fucose. The invention recognizes that by adding fucose to the medium, modulation of the mAb fucosylation was achieved.

### Fucosylation

The invention beneficially recognizes that ADCC activity of the antibody is also influenced by the particular cell line used to produce the antibody. For example, antibodies produced in the mouse myeloma NS/0 cells (or SP2/0 cells) generally have low ADCC, and antibodies produced in rat myeloma YO cells (or YB2/0) cells have high ADCC (Lifely et al., (1995) Glycobiology 5:813-822).

The invention further provides that the type of cell line used for antibody expression affects the carbohydrate structure of the N-linked glycosyl chain, which is attached to the Fc region of the antibody at position corresponding to N297 in IgG1. In certain embodiments, the invention recognizes that the carbohydrate structure of antibodies produced in CHO cells is fucosylated, whereas the carbohydrate chain of antibodies produced in YB2/0 is largely absent of fucose. Antibodies that lack fucose on the carbohydrate structure bind to human FcγRIIIa with higher affinity (Shields et al., (2002) JBC 277:26733-26740). In certain embodiments, anti-CD19 antibodies with variable regions of the invention are characterized by having reduced fucosylation on the N-linked glycosyl chain of the Fc portion of the antibody.

In certain beneficial aspects of the invention, the methods used herein provide that if fucose is absent from the media in which cells are cultured, the CHO cells are unable to synthesize GDP-fucose, and effectively resulting in production of afucosylated antibodies. In effect, the invention provides mechanisms to control the level of fucosylation in the antibodies produced by the methods of the invention.

In certain aspects, the CHO cells used in the invention express a heterologous enzyme that depletes fucose in the cells. Such cells produce afucosylated antibodies. The level of fucosylation may be modulated by fucose supplementation in the medium in which the cells are cultured. In certain embodiments, the heterologous enzyme expressed in the cell is GDP-6-deoxy-D-lyxo-4-hexulose reductase (RMD). RMD is a prokaryotic enzyme that deflects an intermediate in the de novo synthesis of fucose, and hence results in production of afucosylated antibodies.

In certain embodiments, the invention beneficially allows for the control of Fc-fucosylation levels of the antibody.

In the processes involving the production of anti-CD19 antibodies in YB2/0 cells leads to reduce fucosylation. In particular, the YB2/0 cells have reduced fucosylation in the Fc region because the reduced expression of FUT8 enzyme. The level of fucosylation is an important parameter for the anti-CD19 antibodies and it impacts the biological activity of the anti-CD19 antibodies.

In comparison, the methods of the invention advantageously provide an optimal fucosylation pattern of the product. In certain beneficial aspects, the optimal fucosylation product of anti-CD19 antibodies, including VB119 have optimal biological activity.

In certain embodiments, the methods of the invention rely on production of anti-CD19 antibodies using a technology for optimal production and fucosylation of antibodies. In certain embodiments, the technology provides heterologous, stable, cytosolic expression of the bacterial enzyme GDP-4keto-6-deoxy-D-mannose reductase (RMD) in a cell that is intended to be used for production of a glycoprotein or an antibody by subsequent (or simultaneous) transduction of respective genes of the glycoprotein/antibody.

Beneficially, RMD redirects the de-novo fucose synthesis pathway towards a sugar-nucleotide (GDP-Rhamnose) that cannot be metabolized by the cell and moreover acts as an inhibitor of the pathway. Even lowest levels expression of this enzyme is sufficient to block the de novo fucose synthetic pathway almost completely. In certain embodiments, the technology used by methods of the invention is provided included in U.S. Pat. Nos. 7,579,170, 7,931,895, 7,541,029, 8,124,078, 8,153,124, 8,178,093, 8,409,572, 8,642,292, and 8,357,370 which are incorporated by reference in its entirety.

The invention further provides that the level of fucosylation may be modulated by varying the amount of fucose in the cell medium. As an example, Table 2, included below, the level of fucosylation of the exemplary anti-CD19 antibodies and their relative potencies.

TABLE 2
Fucosylation and Relative Potency

| Fucosylation | Relative | EC50 | | Harvest |
|---|---|---|---|---|
| Level (%) | Potency | (ng/mL) | Comment | Days |
| 65 | 41 | 2.0 | With fucose addition | Day 14 |
| 51 | 83 | 3.5 | With fucose addition | Day 15 |
| 49 | 99 | 1.9 | With fucose addition | Day 14 |
| 46 | 105 | 1.2 | With fucose addition | Day 13 |
| 1 | 191 | 1.0 | Without fucose addition | — |
| 2 | 232 | 1.0 | Without fucose addition | — |

As provided above, the methods of the invention provide methods of modulating the level of fucosylation on the anti-CD19 antibody, by adjusting the amount of fucose in the culture medium, and the amount of time that the cells are cultured in such a media.

In certain embodiments, the culture media comprises from about 0.1 mM to about 2.5 mM fucose. In certain embodiments, the culture media comprises from about 0.2 mM to about 2 mM fucose. In certain embodiments, the culture media comprises from about 0.25 mM to about 1 mM fucose. In certain embodiments, the culture media comprises about 0.6 mM fucose. In certain embodiments, the culture media comprises about 0.5 mM fucose. In certain embodiments, the culture media comprises about 0.4 mM fucose. In certain embodiments, the culture media comprises about 0.45 mM fucose. In certain embodiments, the culture media comprises about 0.55 mM fucose.

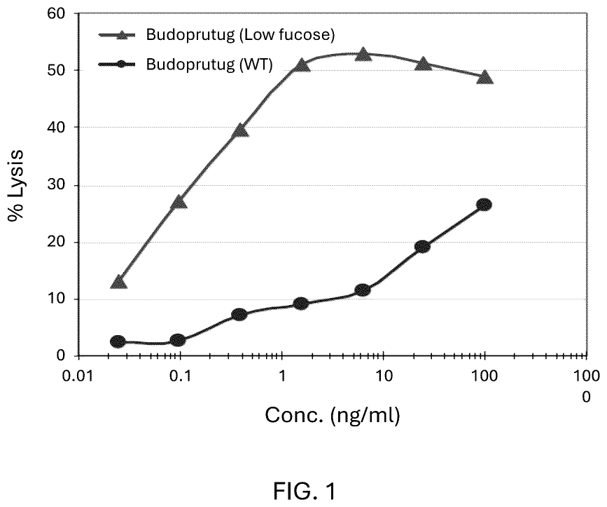

In certain embodiments, the anti-CD19 antibody of the invention, wherein the anti-CD19 antibody of the invention with low fucose concentration has different activity as compared to budoprutug. An example of the activity of ADCC of the low-fucose budoprutug and budoprutug as compared to wt-IgG1 is provided in FIG. 1.

In certain embodiments, the CHO cells for manufacturing anti-CD19 antibodies are cultured from about 5 days to about 30 days. In certain embodiments, the CHO cells for manufacturing anti-CD19 antibodies are cultured for from about 12 days to about 20 days. In certain embodiments, the CHO cells for manufacturing anti-CD19 antibodies are cultured for from about 12 days to about 15 days. In certain embodiments, the CHO cells for manufacturing anti-CD19 antibodies are cultured for about 12 days. In certain embodiments, the CHO cells for manufacturing anti-CD19 antibodies are cultured for about 13 days. In certain embodiments, the CHO cells for manufacturing anti-CD19 antibodies are cultured for about 14 days. In certain embodiments, the CHO cells for manufacturing anti-CD19 antibodies are cultured for about 15 days.

In certain embodiments, the anti-CD19 antibody is about 30% to about 75% fucosylated. In certain embodiments, the anti-CD19 antibody is about 35% to about 70% fucosylated. In certain embodiments, the anti-CD19 antibody is about 40% fucosylated. In certain embodiments, the anti-CD19 antibody is about 45% fucosylated. In certain embodiments, the anti-CD19 antibody is about 50% fucosylated. In certain embodiments, the anti-CD19 antibody is about 55% fucosylated. In certain embodiments, the anti-CD19 antibody is about 60% fucosylated. In certain embodiments, the anti-CD19 antibody is about 65% fucosylated.

In certain aspects, the invention provides that the anti-CD19 antibody has minimal fucosylation. In these embodiments, the invention provides that the anti-CD19 antibodies have less than 5% fucosylation. In certain embodiments, the invention provides that the anti-CD19 antibodies have less than 4% fucosylation. In certain embodiments, the invention provides that the anti-CD19 antibodies have less than 3% fucosylation. In certain embodiments, the invention provides that the anti-CD19 antibodies have less than 2% fucosylation. In certain embodiments, the invention provides that the anti-CD19 antibodies have less than 1% fucosylation.

In certain embodiments, the invention provides that the anti-CD19 antibodies with minimal fucosylation are produced by culturing CHO cells in a media without additional fucose being added to the media.

### Methods of Treatment:

In certain aspects, the invention provides methods of treatment of disease by administration of anti-CD19 antibody, wherein the anti-CD19 antibody is prepared by the methods provided herein.

In certain preferred embodiments, the methods of the invention provide that the disease to be treated with an anti-CD19 antibody of the invention is an autoimmune disease. In certain preferred embodiments, the methods of the invention provide that the disease to be treated with an anti-CD19 antibody of the invention is an autoimmune disorder.

The autoimmune disorder can be a systemic autoimmune disorder or an organ-specific autoimmune disorder. Non-limiting examples of an autoimmune disorder that can be treated using the anti-CD19 antibodies disclosed herein include an acute disseminated encephalomyelitis (ADEM), an Addison's disease, an allergy, allergic rhinitis, an Alzheimer's disease, an anti-phospholipid antibody syndrome (APS), an arthritis such as, e.g., a monoarthritis, an oligoarthritis, or a polyarthritis like an osteoarthritis, a rheumatoid arthritis, a juvenile idiopathic arthritis, a septic arthritis, a spondyloarthropathy, a gout, a pseudogout, or Still's disease, an asthma, an autoimmune deficiency syndrome (AIDS), an autoimmune hemolytic anemia, an autoimmune hepatitis, an autoimmune inner ear disease, a bullous pemphigoid, a celiac disease, a Chagas disease, a chronic obstructive pulmonary disease (COPD), a diabetes mellitus type 1 (IDDM), an endometriosis, a gastrointestinal disorder such as, e.g., an irritable bowel disease or an inflammatory bowel disease like Crohn's disease or an ulcerative colitis, a Goodpasture's syndrome, a Graves' disease, a Guillain-Barré syndrome (GBS), a Hashimoto's thyroiditis, a hidradenitis suppurativa, an idiopathic thrombocytopenia purpura, an interstitial cystitis, a lupus, such as, e.g., a discoid lupus erythematosus, a drug-induced lupus erythematosus, a lupus nephritis, a neonatal lupus, a subacute cutaneous lupus erythematosus, or a systemic lupus erythematosus, a morphea, a multiple sclerosis (MS), a myasthenia gravis, a myopathy such as, e.g., a dermatomyositis, an inclusion body myositis, or a polymyositis, a myositis, a narcolepsy, a neuromyotonia, a Parkinson's disease, a pemphigus vulgaris, a pernicious anemia, a primary biliary cirrhosis, a psoriasis, a recurrent disseminated encephalomyelitis, a rheumatic fever, a schizophrenia, a scleroderma, a Sjögren's syndrome, a skin disorder such as, e.g., dermatitis, an eczema, a statis dermatitis, a hidradenitis suppurativa, a psoriasis, a rosacea or a scleroderma, a tenosynovitis, a uveitis, vasculitis such as, e.g., a Buerger's disease, a cerebral vasculitis, a Churg-Strauss arteritis, a cryoglobulinemia, an essential cryoglobulinemic vasculitis, a giant cell arteritis, a Golfer's vasculitis, a Henoch-Schonlein purpura, a hypersensitivity vasculitis, a Kawasaki disease, a microscopic polyarteritis/polyangiitis, a polyarteritis nodosa, a polymyalgia rheumatica (PMR), a rheumatoid vasculitis, a Takayasu arteritis, or a Wegener's granulomatosis, or a vitiligo. Non-limiting examples of a symptom reduced by a method of treating an autoimmune disorder disclosed herein include inflammation, fatigue, dizziness, malaise, elevated fever and high body temperature, extreme sensitivity to cold in the hands and feet, weakness and stiffness in muscles and joints, weight changes, digestive or gastrointestinal problems, low or high blood pressure, irritability, anxiety, or depression, infertility or reduced sex drive (low libido), blood sugar changes, and depending on the type of autoimmune disease, an increase in the size of an organ or tissue, or the destruction of an organ or tissue. Non-limiting examples of an inflammation symptom reduced by a method of treating an autoimmune disorder disclosed herein include edema, hyperemia, erythema, bruising, tenderness, stiffness, swollenness, fever, a chill, congestion of the respiratory tract including nose, and bronchi, congestion of a sinus, a breathing problem, fluid retention, a blood clot, a loss of appetite, an increased heart rate, a formation of granulomas, fibrinous, pus, or non-viscous serous fluid, a formation of an ulcer, or pain.

In certain embodiments, the autoimmune disease or disorder is primary membranous nephropathy (PMN). Primary membranous nephropathy (PMN) is a kidney-specific, autoimmune glomerular disease that presents with increased protein in the urine associated with a pathognomonic pattern of injury in glomeruli. PMN is the commonest cause of idiopathic nephrotic syndrome in nondiabetic adults worldwide, representing between 20% and 37% in most series and rising to as high as 40% in adults over 60. In certain embodiments, the invention provides that the anti-CD19 antibodies prepared using the methods of the invention are administered for the treatment of PMN.

In certain embodiments, the methods of the invention provide that the disease to be treated with an anti-CD19 antibody of the invention is cancer. In certain embodiments, the cancer could be any cancer caused by B cells, such as B cell lymphoma.

### Methods of Administration:

The antibodies of the invention are preferably used to treat patients with B cell disorders such as autoimmune disorders with a B cell component such as rheumatoid arthritis, myasthenia gravis, multiple sclerosis, systemic lupus erythematosus.

In the case of antibodies directed against CD19, it is sometimes useful to clear the normal B cells from the body, as these cells are likely to titrate the antibody of the invention. Rituxan™ may be used for this purpose, according to standard procedures. Alternatively, the anti-CD 19 antibodies of the invention may be used to clear the normal B cells from the body.

In certain embodiments, the invention provides that the antibodies prepared by the methods of the invention may be administered parenterally. In certain embodiments, the invention provides that the anti-CD19 antibodies of the invention may be administered as an intravenous infusion. Other methods of administration include injection routes such as subcutaneous, intradermal, intramuscular, intraperitoneal, or intravenous (bolus) delivery. Inhalation and oral delivery are also possible methods of delivery.

For a 70 kilogram human, a typical dose is in the range of about 50 milligrams to 2 grams, with a preferred dose in the range of about 400-600 milligrams. Dosing may be repeated about once every three to six weeks.

Pharmaceutical compositions of the invention may be used in the form of solid, semisolid, or liquid dosage forms, such as, for example, pills, capsules, powders, liquids, suspensions, or the like, preferably in unit dosage forms suitable for administration of precise dosages. The compositions include a conventional pharmaceutical carrier or excipient and, in addition, may include other medicinal agents, pharmaceutical agents, carriers, adjuvants, etc. Such excipients may include other proteins, such as, for example, human serum albumin or plasma proteins. Actual methods of preparing such dosage forms are known or will be apparent to those skilled in the art. The composition or formulation to be administered will, in any event, contain a quantity of the active component(s) in an amount effective to achieve the desired effect in the subject being treated.

Administration of the compositions hereof can be via any of the accepted modes of administration for agents that exhibit such activity. These methods local or systemic administration. Intravenous injection in a pharmaceutically acceptable carrier is a preferred method of administration. The amount of active compound administered will, of course, be dependent on the subject being treated, the severity of the affliction, the manner of administration, and the judgment of the prescribing physician.

### Compositions for Production:

In certain aspects, the present invention provides a composition for preparation of anti-CD19 antibody. In certain embodiments, the invention provides a composition for preparation of an anti-CD19 antibody, wherein the anti-CD19 antibody comprises variable domain comprising a heavy chain variable region that is at least 90% identical to SEQ ID NO: 13 and has an amino acid substitution at one or more residues corresponding to Gln5, Arg19, Leu20, Arg40, Gln43, Lys65, Ser85, Ser88, and Val93, and the anti-CD19 antibody is fucosylated; and the composition comprises a cell growth media for Chinese Hamster Ovary (CHO) cells and fucose.

In certain embodiments, the invention provides a composition for preparation of an anti-CD19 antibody, wherein the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and the anti-CD19 antibody is fucosylated; and the composition comprises a cell growth media for Chinese Hamster Ovary (CHO) cells and fucose.

In certain embodiments, the invention provides a composition for preparation of an anti-CD19 antibody, wherein the anti-CD19 antibody comprises the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53, and Leu53; and the anti-CD19 antibody is fucosylated and the composition comprises a cell growth media for Chinese Hamster Ovary (CHO) cells and fucose.

In certain embodiments, the CHO cells are CHO DG44 cells. In certain embodiments, the process does not comprise the use of live viruses and/or infectious retroviral particles. In certain embodiments,

In certain embodiments, the culture media comprises from about 0.1 mM to about 2.5 mM fucose. In certain embodiments, the culture media comprises from about 0.2 mM to about 2 mM fucose. In certain embodiments, the culture media comprises from about 0.25 mM to about 1 mM fucose. In certain embodiments, the culture media comprises about 0.6 mM fucose. In certain embodiments, the culture media comprises about 0.5 mM fucose. In certain embodiments, the culture media comprises about 0.4 mM fucose. In certain embodiments, the culture media comprises about 0.45 mM fucose. In certain embodiments, the culture media comprises about 0.55 mM fucose.

The table below provides the sequences for nucleic acids, proteins, and peptides discussed herein.

| SEQ ID NO: | Sequence |
|---|---|
| 1 | CAGGTGCAACTGCAGCAGCCTGGGGCTGAAGTGGTGAAGCCTGGGGCTT |
| | CAGTGAGACTGTCCTGCAAGACTTCTGGCTACACCTTCACCAGCAACTG |
| | GATGCACTGGGTGAAGCAGAGGCCTGGACAAGGCCTTGAGTGGATCGG |
| | AGAGATTGATCCTTCTGATAGTTATACTAACTACAATCAAAAGTTCAAG |
| | GGCAAGGCCAAGTTGACTGTAGACAAATCCTCCAGCACAGCCTACATGG |
| | AAGTCAGCAGCCTGACATCTGAGGACTCTGCGGTCTATTACTGTGCAAG |
| | AGGTAGCAACCCTTACTACTATGCTATGGACTACTGGGGTCAAGGAACC |
| | TCAGTCACCGTCTCCTCA |
| 2 | CAGGTGCAACTGCAGCAGCCTGGGGCTGAAGTGGTGAAGCCTGGGGCTT |
| | CAGTGAGACTGTCCTGCGAGACTTCTGGCTACACCTTCACCAGCAACTG |
| | GATGCACTGGGTGAAGCAGAGGCCTGACCAAGGACTTGAGTGGATCGG |
| | AGAGATTGATCCTTCTGATAGTTATACTAACTACAATCAAAAGTTCAAG |
| | GGCAAGGCCGAATTGACTGTAGACAAATCCTCCAGCACAGCCTACATGG |
| | AAGTCAGCGACCTGACATCTGAGGACTCTGCGGTCTATTACTGTGCAAG |
| | AGGTAGCAACCCTTACTACTATGCTATGGACTACTGGGGTCAAGGAACC |
| | TCAGTCACCGTCTCCTCA |
| 3 | CAGGTGCAACTGCAGCAGCCTGGGGCTGAAGTGGTGAAGCCTGGGGCTT |
| | CAGTGAGACTGTCCTGCAAGACTTCTGGCTACACCTTCACCAGCAACTG |
| | GATGCACTGGGTGAAGCAGAGACCTGGACAAGGACTTGAGTGGATCGG |
| | AGAGATTGATCCTTCTGATAGTTATACTAACTACAATCAAAAGTTCAAG |
| | GGCAAGGCCGAATTGACTGTAGACAAATCCTCCAGCACAGCCTACATGG |
| | AAGTCAGCGACCTGACATCTGAGGACTCTGCGGTCTATTACTGTGCAAG |
| | AGGTAGCAACCCTTACTACTATGCTATGGACTACTGGGGTCAAGGAACC |
| | TCAGTCACCGTCTCCTCA |
| 4 | CAGGTGCAACTGGAGCAGCCTGGGGCTGAAGTGAAGAAGCCTGGGGCT |
| | TCAGTGAAGGTGTCCTGCAAGGCTTCTGGCTACACCTTCACCAGCAACT |
| | GGATGCACTGGGTGAAGCAGAGGCCTGGACAAGGACTTGAGTGGATCG |
| | GAGAGATTGATCCTTCTGATAGTTATACTAACTACAATCAAAAGTTCAA |
| | GGGCAAGGCCAAGTTGACTGTAGACAAATCCTCCAGCACAGCCTACATG |
| | GAAGTCAGCGACCTGACAGCTGAGGACTCTGCGGTCTATTACTGTGCAA |
| | GAGGTAGCAACCCTTACTACTATGCTATGGACTACTGGGGTCAAGGAAC |
| | CTCAGTCACCGTCTCCTCA |
| 5 | CAGGTGCAACTGGAGCAGCCTGGGGCTGAAGTGGTGAAGCCTGGGGCTT |
| | CAGTGAAGGTGTCCTGCAAGACTTCTGGCTACACCTTCACCAGCAACTG |
| | GATGCACTGGGTGAAGCAGACGCCTGGAAAAGGACTTGAGTGGATCGG |
| | AGAGATTGATCCTTCTGATAGTTATACTAACTACAATCAAAAGTTCGAT |
| | GGCAAGGCCAAGTTGACTGTAGACAAATCCTCCAGCACAGCCTACATGG |
| | AAGTCAGCGACCTGACAGCTGAGGACTCTGCGACCTATTACTGTGCAAG |
| | AGGTAGCAACCCTTACTACTATGCTATGGACTACTGGGGTCAAGGAACC |
| | TCAGTCACCGTCTCCTCA |
| 6 | CAGGTGCAACTGGAGCAGCCTGGGGCTGAAGTGAAGAAGCCTGGGGCT |
| | TCAGTGAAGGTGTCCTGCAAGGCTTCTGGCTACACCTTCACCAGCAACT |
| | GGATGCACTGGGTGAGACAGGCACCTGGAAAAGGACTTGAGTGGATCG |
| | GAGAGATTGATCCTTCTGATAGTTATACTAACTACAATCAAAAGTTCGA |
| | TGGCAAGGCCAAGTTGACTGTAGACAAATCCTCCAGCACAGCCTACATG |
| | GAAGTCAGCGACCTGACATCTGAGGACTCTGCGACCTATTACTGTGCAA |
| | GAGGTAGCAACCCTTACTACTATGCTATGGACTACTGGGGTCAAGGAAC |
| | CTCAGTCACCGTCTCCTCA |
| 7 | CAGGTGCAACTGGAGCAGCCTGGGGCTGAAGTGGTGAAGCCTGGGGCTT |
| | CAGTGAAGGTGTCCTGCAAGACTTCTGGCTACACCTTCACCAGCAACTG |
| | GATGCACTGGGTGAAGCAGAGGCCTGGACAAGGACTTGAGTGGATCGG |
| | AGAGATTGATCCTTCTGATAGTTATACTAACTACAATCAAAAGTTCGAT |
| | GGCAAGGCCAAGTTGACTGTAGACAAATCCTCCAGCACAGCCTACATGG |
| | AAGTCAGCGACCTGACATCTGAGGACTCTGCGACCTATTACTGTGCAAG |
| | AGGTAGCAACCCTTACTACTATGCTATGGACTACTGGGGTCAAGGAACC |
| | TCAGTCACCGTCTCCTCA |
| 8 | CAAATTGTTCTCACCCAGTCTCCAGCAATCATGTCTGCATCTCCAGGGGA |
| | GAAGGTCACCATGACCTGCAGTGCCAGCTCAGGTGTCAACTACATGCAC |
| | TGGTACCAGCAGAAGCCAGGCACCTCCCCCAAAAGATGGATTTATGACA |
| | CATCCAAACTGGCTTCTGGAGTCCCTGCTCGCTTCAGTGGCAGTGGGTCT |
| | GGGACCTCTTATTCTCTCACAATCAGCAGCATGGAGGCTGAAGATGCTG |
| | CCACTTATTACTGCCATCAGCGAGGTAGTTACACGTTCGGAGGGGGGAC |
| | CAAGCTGGAAATAAA |
| 9 | CAAATTGCTCTCACCCAGGAGCCAGCAATCATGTCTGCATCTCCAGGGG |
| | AGAAGGTCACCATGACCTGCAGTGCCAGCTCAGGTGTCAACTACATGCA |
| | CTGGTATCAGCAGAAGCCAGGCACCTCCCCCAAAAGATGGATTTATGAC |
| | ACATCCAAACTGGATTCTGGAGTCCCTGCTCGCTTCAGTGGCAGTGGGT |
| | CTGGGACCTCTTATTCTCTCACAATCAGCAGCATGGAGGCTGAAGATGC |
| | TGCCACTTATTACTGCCATCAGCGAGGTAGTTACACGTTCGGAGGGGGG |
| | ACCAAGCTGGAAATAAA |
| 10 | GACATTGTTCTCACCCAGTCTCCAGCAACTTTGTCTGCATCTCCAGGGGA |
| | GAAGGTCACCATGACCTGTAGTGCCAGCTCAGGTGTCAACTACATGCAC |
| | TGGTATCAGCAGAAGCCAGGCACCTCCCCCAAAAGATGGATTTATGACA |
| | CATCCAAACTGGATTCTGGAGTCCCTGCTCGCTTCAGTGGCAGTGGGTCT |
| | GGGACCTCTTATTCTCTCACAATCAGCAGCATGGAGGCTGAAGATGCTG |
| | CCACTTATTACTGCCATCAGCGAGGTAGTTACACGTTCGGAGGGGGGAC |
| | CAAGCTGGAAATAAA |
| 11 | CAAATTGTTCTCACCCAGTCTCCAGCAACTTTGTCTGCATCTCCAGGGGA |
| | GAAGGCTACCATGACCTGCAGTGCCAGCTCAGGTGCTAACTACATGCAC |
| | TGGTACCAGCAGAAGCCAGGCACCTCCCCCAAAAGATGGATTTATGACA |
| | CATCCAAACTGGCTTCTGGAGTCCCTGCTCGCTTCAGTGGCAGTGGGTCT |
| | GGGACCTCTTATTCTCTCACAATCGAGAGCATGGAGGCTGAAGATGCTG |
| | CCACTTATTACTGCCATCAGCGAGGTAGTTACACGTTCGGAGGGGGGAC |
| | CAAGCTGGAAATAAA |
| 12 | CAAATTGTTCTCACCCAGTCTCCAGCAACTTTGTCTGCATCTCCAGGGGA |
| | GAAGGCTACATGACCTGTAGTGCCAGCTCAGGTGTCAACTACATGCACT |
| | GGTACCAGCAGAAGCCAGGCACCTCCCCCAAAAGATGGATTTATGACAC |
| | AGACAAAACGGCTTCTGGAGTCCCTGCTCGCTTCAGTGGCAGTGGGTCT |
| | GGGACCTCTTATTCTCTCACAATCAGCAGCATGGAGGCTGAAGATGCTG |
| | CCACTTATTACTGCCATCAGCGAGGTAGTTACACGTTCGGAGGGGGGAC |
| | CAAGCTGGAAATAAA |
| 13 | QVQLQQPGAEVVKPGASVRLSCKTSGYTFTSNWMHWVKQRPGQGLEWIG |
| | EIDPSDSYTNYNQKFKGKAKLTVDKSSSTAYMEVSSLTSEDSAVYYCARGS |
| | NPYYYAMDYWGQGTSVTVSS |
| 14 | QVQLQQPGAEVVKPGASVRLSCETSGYTFTSNWMHWVKQRPDQGLEWIG |
| | EIDPSDSYTNYNQKFKGKAELTVDKSSSTAYMEVSDLTSEDSAVYYCARGS |
| | NPYYYAMDYWGQGTSVTVSS |
| 15 | QVQLQQPGAEVVKPGASVRLSCKTSGYTFTSNWMHWVKQRPGQGLEWIG |
| | EIDPSDSYTNYNQKFKGKAELTVDKSSSTAYMEVSDLTSEDSAVYYCARGS |
| | NPYYYAMDYWGQGTSVTVSS |
| 16 | QVQLEQPGAEVKKPGASVKVSCKASGYTFTSNWMHWVKQRPGQGLEWIG |
| | EIDPSDSYTNYNQKFKGKAKLTVDKSSSTAYMEVSDLTAEDSAVYYCARGS |
| | NPYYYAMDYWGQGTSVTVSS |
| 17 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSS |
| 18 | QVQLEQPGAEVKKPGASVKVSCKASGYTFTSNWMHWVRQAPGKGLEWIG |
| | EIDPSDSYTNYNQKFDGKAKLTVDKSSSTAYMEVSDLTSEDSATYYCARGS |
| | NPYYYAMDYWGQGTSVTVSS |
| 19 | QVQLEQPGAEVVKPGASVKVSCKTSGYTFTSNWMHWVKQRPGQGLEWIG |
| | EIDPSDSYTNYNQKFDGKAKLTVDKSSSTAYMEVSDLTSEDSATYYCARGS |
| | NPYYYAMDYWGQGTSVTVSS |
| 20 | QVQLQQPGAEVKKPGASVRLSCETSGYTFTSNWMHWVKQRPDQGLEWIG |
| | EIDPSDSYTNYNQKFDGKAELTVDKSSSTAYMEVSDLTSEDSAVYYCARGS |
| | NPYYYAMDYWGQGTSVTVSS |
| 21 | QVQLEQPGAEVKKPGASVKVSCKASGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSS |
| 22 | QVQLXQPGAEVXKPGASVXXSCXXSGYTF |
| 23 | WVXQXPXXGLEWIG |
| 24 | YNQKFXGKAXLTVDKSSSTAYMEVSXLTXEDSAXYYCA |
| 25 | QIVLTQSPAIMSASPGEKVTMTCSASSGVNYMHWYQQKPGTSPKRWIYDTS |
| | KLASGVPARFSGSGSGTSYSLTISSMEAEDAATYYCHQRGSYTFGGGTKLEI |
| | K |
| 26 | QIALTQEPAIMSASPGEKVTMTCSASSGVNYMHWYQQKPGTSPKRWIYDTS |
| | KLDSGVPARFSGSGSGTSYSLTISSMEAEDAATYYCHQRGSYTFGGGTKLEI |
| | K |
| 27 | DIVLTQSPATLSASPGEKVTMTCSASSGVNYMHWYQQKPGTSPKRWIYDTS |
| | KLDSGVPARFSGSGSGTSYSLTISSMEAEDAATYYCHQRGSYTFGGGTKLEI |
| | K |
| 28 | QIVLTQSPATLSASPGEKATMTCSASSGANYMHWYQQKPGTSPKRWIYDTS |
| | KLASGVPARFSGSGSGTSYSLTIESMEAEDAATYYCHQRGSYTFGGGTKLEI |
| | K |
| 29 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIK |
| 30 | QIALTQEPAIMSASPGEKATMTCSASSGVNYMHWYQQKPGTSPKRWIYDTS |
| | KLDSGVPARFSGSGSGTSYSLTIESMEAEDAATYYCHQRGSYTFGGGTKLEI |
| | K |
| 31 | QIVLTQSPATLSASPGEKATMTCSASSGANYMHWYQQKPGTSPKRWIYDT |
| | DKTASGVPARFSGSGSGTSYSLTIESMEAEDAATYYCHQRGSYTFGGGTKL |
| | EIK |
| 32 | XIXLTQXPAXXSASPGEKXTMTC |
| 33 | DTXKXX |
| 34 | QVQLQQPGAEVVKPGASVRLSCKTSGYTFTSNWMHWVKQRPGQGLEWIG |
| | EIDPSDSYTNYNQKFKGKAKLTVDKSSSTAYMEVSSLTSEDSAVYYCARGS |
| | NPYYYAMDYWGQGTSVTVSS |
| 35 | QIVLTQSPAIMSASPGEKVTMTCSASSGVNYMHWYQQKPGTSPKRWIYDTS |
| | KLASGVPARFSGSGSGTSYSLTISSMEAEDAATYYCHQRGSYTFGGGTKLEI |
| | K |
| 36 | VRLSCKTSG |
| 37 | WVKQRPGQG |
| 38 | YNQKFKGKA |
| 39 | FKGKAKLTV |
| 40 | YMEVSSLTS |
| 41 | VYYCARGSN |
| 42 | IVLTQSPAI |
| 43 | VLTQSPAIM |
| 44 | VTMTCSASS |
| 45 | VNYMHWYQQ |
| 46 | WIYDTSKLA |
| 47 | IYDTSKLAS |
| 48 | CGTAAGTGGATCC |
| 49 | GCTAGCTCCAGC |
| 50 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 51 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 52 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |
| 53 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |
| 54 | QVQLEQPGAEVVKPGASVKVSCKTSGYTFTSNWMHWVKQTPGKGLEWIG |
| | EIDPSDSYTNYNQKFDGKAKLTVDKSSSTAYMEVSDLTAEDSATYYCARGS |
| | NPYYYAMDYWGQGTSVTVSSASTKGPSVFPLAPSSKSTSGGTAALGCLVK |
| | DYFPEPVTVSWNSGALTSGVHTFPAVLQSSGLYSLSSVVTVPSSSLGTQTYI |
| | CNVNHKPSNTKVDKRVEPKSCDKTHTCPPCPAPELLGGPSVFLFPPKPKDTL |
| | MISRTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 55 | QIVLTQSPATLSASPGEKATMTCSASSGVNYMHWYQQKPGTSPKRWIYDT |
| | DKTASGVPARFSGSGSGTSYSLTISSMEAEDAATYYCHQRGSYTFGGGTKL |
| | EIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTK |
| | SFNRGEC |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A process for preparation of an anti-CD19 antibody, wherein:
the anti-CD19 antibody comprises variable domain comprising a heavy chain variable region that is at least 90% identical to SEQ ID NO: 13 and has an amino acid substitution at one or more residues corresponding to Gln5, Arg19, Leu20, Arg40, Gln43, Lys65, Ser85, Ser88, and Val93; and
the process comprises production of the anti-CD19 antibody in Chinese Hamster Ovary (CHO) cells.

2. The process for preparation of an anti-CD19 antibody, wherein:
the anti-CD19 antibody comprises variable domain comprising a light chain variable region that is at least 90% identical to SEQ ID NO: 25 and has an amino acid substitution at one or more residues corresponding to Ile10, Met11, Val19, Ser51, and Leu53; and
the process comprises production of the anti-CD19 antibody in Chinese Hamster Ovary (CHO) cells.

3. The process for preparation of an anti-CD19 antibody, wherein:
the anti-CD19 antibody comprises SEQ ID NO: 13 with an amino acid substitution at one or more residues corresponding to Gln5, Arg19, Leu20, Arg40, Gln43, Lys65, Ser85, Ser88, and Val93, and SEQ ID NO: 25 with an amino acid substitution at one or more residues corresponding to Ile10, Met11, Val19, Ser51, and Leu53; and
the process comprises production of the anti-CD19 antibody in Chinese Hamster Ovary (CHO) cells.

4. The process of claim 1, wherein the anti-CD19 antibody variable domain comprises a heavy chain variable region of SEQ ID NO: 17 and a light chain variable region of SEQ ID NO: 29.

5. The process of claim 1, wherein the CHO cells are CHO DG44 cells.

6. The process of claim 1, wherein the process does not comprise the use of live viruses and/or infectious retroviral particles.

7. The process of claim 1, wherein the process comprises one or more of the below steps:
thawing the CHO cells;
culturing the thawed CHO cells;
harvest the cultured CHO cells;
isolate the anti-CD19 antibody; and
purify the anti-CD19 antibody.

8. The process of claim 1, wherein the heavy chain variable region having, compared to SEQ ID NO:13, one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

9. The process of claim 1, wherein the heavy chain variable region is SEQ ID NO: 13 comprising an amino acid substitution at one or more residues corresponding to Gln5, Arg19, Leu20, Arg40, Gln43, Lys65, Ser85, Ser88, and Val93.

10. The process of claim 1, wherein the heavy chain variable region has one or more amino acid substitutions selected from the group consisting of Gln5Glu, Arg19Lys, Leu20Val, Arg40Thr, Gln43Lys, Lys65Asp, Ser85Asp, Ser88Ala, and Val93Thr.

11. The process of claim 1, wherein the heavy chain variable region is the amino acid sequence of SEQ ID NO: 17.

12. The process of claim 1, wherein the anti-CD19 antibody variable domain further comprises a light chain variable region that is at least 90% identical to SEQ ID NO: 25 and has an amino acid substitution at one or more residues corresponding to Ile10, Met11, Val19, Ser51, and Leu53.

13. The process of claim 1, wherein the light chain variable region is at least 95% identical to SEQ ID NO: 25.

14. The process of claim 2, wherein the light chain variable region has one or more amino acid substitutions selected from the group consisting of Ile10Thr, Met11Leu, Val19Ala, Ser51Asp, and Leu53Thr.

15. The process of claim 2, wherein the light chain variable region is SEQ ID NO: 25 comprising an amino acid substitution at one or more residues corresponding to Ile10, Met11, Val19, Ser51, and Leu53.

16. The process of claim 2, wherein the light chain variable region has one or more amino acid substitutions selected from the group consisting of Ile10Thr, Met11Leu, Val19Ala, Ser51Asp, and Leu53Thr.

17. The process of claim 2, wherein the light chain variable region is the amino acid sequence of SEQ ID NO: 29.

18. The process of claim 3, wherein the heavy chain variable region comprises one or more of substitutions Gln5Glu, Arg19Lys, Leu20Val, Arg40Thr, Gln43Lys, Lys65Asp, Ser85Asp, Ser88Ala, and Val93Thr.

19. The process of claim 3, wherein the light chain variable region comprises one or more of substitutions Ile10Thr, Met11Leu, Val19Ala, Ser51Asp, and Leu53Thr.
